(19) United States
(12) Patent Application Publication
(10) Pub. No.: US 2026/0097859 A1
(43) Pub. Date: Apr. 9, 2026

(54) AIRCRAFT BLEED AIR CONTAMINANT SENSOR SYSTEM

(71) Applicant: HarcoSemco LLC, Branford, CT (US)

(72) Inventors: Robert Croce, Branford, CT (US); Richard Fox, Queen Creek, AZ (US)

(21) Appl. No.: 19/406,268

(22) Filed: Dec. 2, 2025

Related U.S. Application Data
(63) Continuation of application No. PCT/US2024/032297, filed on Jun. 3, 2024.
(60) Provisional application No. 63/505,766, filed on Jun. 2, 2023.

(51) Int. Cl. B64D 43/00; G01N 33/00

(52) U.S. Cl. CPC B64D 43/00; G01N 33/0073

(57) ABSTRACT

Provided is a sensing assembly including a housing defining a cavity, a first printed circuit board (PCB) disposed in the cavity, sensing circuitry disposed on the first PCB for detecting particles of various sizes, an inlet in communication with the cavity upstream of the first PCB, and an outlet in communication with the cavity downstream of the first PCB, wherein the inlet and outlet are sized to create airflow across the first PCB.

### RELATED APPLICATIONS

This application is a continuation of International Application No. PCT/US2024/032297, filed Jun. 3, 2024, which claims the benefit of U.S. Provisional Application No. 63/505,766 filed Jun. 2, 2023, which is hereby incorporated herein by reference.

### FIELD OF INVENTION

The present invention relates generally to a sensor system, and more particularly to a bleed air sensor system for an aircraft.

### BACKGROUND

Aircraft air supply systems include pneumatic air delivered to the air conditioning system from propulsion engines and auxiliary power units (APU). The air typically passes through an ozone converter to remove ozone or hydrocarbon contaminants, prior to entering the air conditioning system. Air exiting the air conditioning system may contain contaminants that have condensed within the air conditioning system and are released when the heat exchangers in the system are heated. Aerosol from contaminants such as trace turbine oil, hydraulic oil, and de-icing fluid, or ingested exhaust from airport operations, are then released into the cabin where passengers and flight crew become exposed.

Contaminants originating from the propulsion engine on-ground include traces of oil that may have leaked out of engine oil seals while the engine was stationary with no buffer air pressure on the seals to prevent oil intrusion to the air side of the seal, ingested fuel vapors and exhaust fumes, and deicing fluid. Primary sources of contamination from the APU are the most likely source for ingestion of turbine oil ingestion from the APU bay, hydraulic fluid collected around the APU inlet, and deicing fluid sprayed into the APU inlet. The aerosols entrained by air from the engine and APU condense in the air conditioning heat exchangers and are vaporized into the air conditioning air when the heat exchangers during normal operation.

### SUMMARY OF INVENTION

According to an aspect, a sensing assembly is provided including a housing defining a cavity, a first printed circuit board (PCB) disposed in the cavity, sensing circuitry disposed on the first PCB for detecting particles of various sizes, an inlet in communication with the cavity upstream of the first PCB, and an outlet in communication with the cavity downstream of the first PCB, wherein the inlet and outlet are sized to create airflow across the first PCB.

According to another aspect, a sensing assembly is provided including a housing defining a cavity, sensing circuitry disposed in the cavity for detecting particles of various sizes, the sensing circuitry includes a particulate matter sensor, a particle sensor, a carbon dioxide sensor, a gas sensor, and an environmental parameters sensor, an inlet in communication with the cavity upstream of the sensing circuitry, and an outlet in communication with the cavity downstream of the sensing circuitry, wherein the inlet and outlet are sized to create airflow across the sensing circuitry.

According to still another aspect, a method of sensing a source of contaminants from a bleed air source in an aircraft, such as pneumatic sources, temperature probe ports, gasper systems, mix manifolds, and return air filters is provided. The method includes receiving the bleed air at an inlet, delivering the bleed air across one or more sensors, including a particulate matter sensor, a particle sensor, a carbon dioxide sensor, a gas sensor, and an environmental parameters sensor, and delivering the bleed air to an outlet.

The foregoing and other features of the application are described below with reference to the drawings.

### BRIEF DESCRIPTION OF THE DRAWINGS

FIG. 1 is a schematic view of a sensing system.

FIG. 2 is a perspective view of an exemplary sensor assembly.

FIG. 3 is a cross-sectional view of an inlet port of the sensor assembly.

FIG. 4 is a top view of another exemplary sensor assembly.

FIG. 5 is a perspective view of the sensor assembly

FIG. 6 is a perspective view of a printed circuit board of the sensor assembly.

### DETAILED DESCRIPTION

The principles of the present application relate to a sensor system, such as a bleed air sensor system for an aircraft, and thus will be described below in this context. It will be appreciated that the principles of the application may be applicable to other sensor systems, such as sensor systems for other vehicles.

In commercial aircraft systems, the engine and APU air is typically supplied to pneumatic ducts at pressure ranging from approximately twenty to forty-five PSIG, depending on the engine and APU operating conditions. Downstream of the air conditioning packs, the pressure of the air entering the cabin has been further reduced to around five tenths to one PSIG. Sample air may be taken from various locations within the aircraft, such as pressurization off-takes for ported locations such as hydraulic systems, water tanks, trim air for cabin air-conditioning temperature regulation, ozone converters, primary pack heat exchanger, etc. The sample air allows for the identification of contaminants and contaminant sources so that an airline may locate and eliminate the contaminants and reduced aircraft downtime.

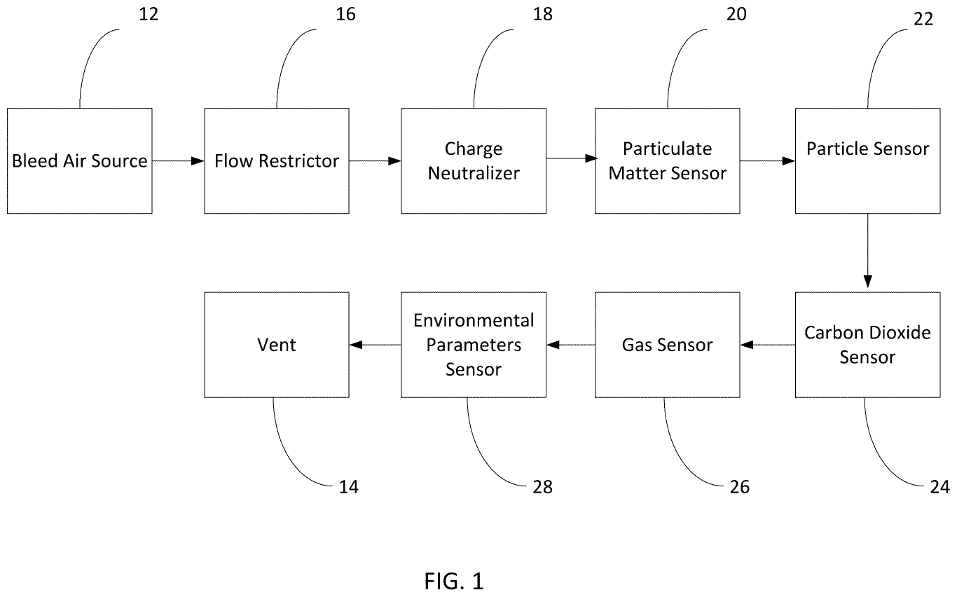

Turning initially to FIG. 1, a sensing system for sensing the source of contaminants is shown generally at reference numeral 10. The sensing system 10 is configured to received air from a cabin supply air source 12, such as from pneumatic sources, temperature probe ports, gasper systems, mix manifolds, return air filters, etc., and after sensing, deliver the air to a vent 14 to provide constant airflow across the sensing elements. The system can sense, for example, particulate matter, ultrafine particles, carbon dioxide, carbon monoxide, and volatile organic compounds originating from common aircraft fluids such as turbine oil, hydraulic fluid, deicing fluid and exhaust. The system thereby provides operators meaningful information for sensing air pollution to provide health and safety benefits to passengers and crew.

The sensing system may include a flow restrictor 16, a charge neutralizer 18, a particulate matter sensor 20, a particle sensor 22, a carbon dioxide sensor 24, a gas sensor, such as a Volatile Organic Compound (VOC) sensor 26, and an environmental parameters sensor 28. It will be appreciated that one or more flow restrictor 16, charge neutralizer 18, particulate matter sensor 20, particle sensor 22, carbon dioxide sensor 24, VOC sensor 26, and environmental parameters sensor 28 may be provided, and that each element may include multiple elements. The air flow channel in the system may be sized to minimize purge time across the sensors while not disturbing the air flow across the particle sensors. An internal battery may be provided to maintain the sensors in a state of readiness and to maintain the ionization field when the aircraft electrical system is powered down.

Referring now to the flow restrictor 16, the flow restrictor is provided to generate laminar flow of the bleed air to less than one meter per second to provide a repeatable flow environment to the other elements in the sensing system.

Referring now to the charge neutralizer 18 or ionization particle neutralizer, the charge neutralizer 18 is configured to be positioned at an inlet of the sensing system 10 to receive the bleed air from the flow restrictor 16 and to remove static charge from the particles and reduce attraction of particles to the walls of the sensing system housing, which themselves may be made of a conductive metal or conductive extruded material that may be grounded to prevent/reduce static buildup. The charge neutralizer thereby ensures that aerosols transit into the housing and to the downstream sensors. In an implementation, the charge neutralizer 18 may be placed a predetermined distance away from a center of the bleed air stream entering the housing, for example around three quarters of an inch to around one inch away, and on a bottom or side of the housing for example. The charge neutralizer 18 may be a strip of Polonium 210 or similar ionization removal strip, for example as a single or dual strip, which increases the life of the charge neutralizer 18. In an implementation, the charge neutralizer 18 may be removed from the system.

Referring now to the particulate matter sensor 20, the particulate matter sensor 20 is provided to detect and quantify particles, such as fine particles and very small particles in the flow stream of hydraulic fluid, atmospheric contaminants, etc. The particulate matters sensor 20 may be an optical spectroscopy sensor, such as an optical light emitting diode (LED) sensor, such as one or more an infrared LED sensors that senses particles down to about three hundred ten nanometers, one or more blue LED sensors that senses particles down to about two hundred twenty-five nanometers, and one or more ultraviolet LED sensors that senses the smaller nanometer size range ultrafine particles, such as particles down to about fifty nanometers. The sensors may detect a particle concentration around zero to 100,000,000 particles and ultrafine particles per cubic centimeter.

Referring now to the particle sensor 22, the particle sensor is provided to detect and quantify particles in the flow stream down to about two nanometers, for example turbine oils, hydraulic fluids, engine exhaust, etc. The particle sensor may be at least one ionization sensor, or one ultraviolet LED or other ultraviolet light source sensor. In an implementation with one ionization sensor, the sensor may include a built-in reference electrode with a single source or a single electrode with two separate sources. The reference electrode electrically accounts for variation in humidity and other non-aerosol effects, such as altitude and temperature. In an implementation with two ionization sensors, one ionization sensor may be provided as a reference sensor and one ionization sensor may be provided as a sample sensor such that the sensors are exposed to the same temperature and pressure fluctuations with the reference sensor not being directly influenced from the flow or humidity. Ultrafine particles tend to condense and grow as they pass through the aircraft bleed and environmental control system, so the full range of particle measurement enables sensing at various locations within the ductwork to capture the presence of particles and ultrafine particles.

Referring now to the carbon dioxide sensor 24, the carbon dioxide sensor 24 may be provided to monitor the presence of exhaust ingestion, which may be indicated by a rapid rise and fall over a period of fifteen to thirty seconds for example. An air sample within the aircraft mix manifold area may be similar to air in the cabin, and will have a higher carbon dioxide level from the aircraft occupants or from dry ice sublimation. Exhaust ingestion measurements are differentiated from cabin sourced carbon dioxide by means of the algorithm that verifies movement of the aircraft and a rate of rise of the carbon dioxide signal. Increase in carbon dioxide from occupants or dry ice sublimation will be gradual, while an increase in carbon dioxide from exhaust ingestion will be rapid and drop off more quickly.

Referring now to the gas sensor 26, the gas sensor 26, which may have artificial intelligence incorporated therein for identifying contaminant type, may be provided to detect and quantify contaminants in the bleed air such as carbon monoxide, turbine oil, hydraulic fluid, deicing fluid, ground exhaust, engine exhaust, etc. The gas sensor 26, or metal oxide semiconductor sensor, may be employed with artificial intelligence to respond to the contaminants. In aircrafts, carbon monoxide levels are normally less than three parts per million, and the presence of higher levels may indicate ingestion of exhaust from an external source. For example, the presence of carbon monoxide in the mix manifold sensors may be from exhaust ingestion, or potentially an early indication of fire. Aircrafts primarily utilize 3000 psi hydraulic fluids and 5000 psi hydraulic fluids. The gas sensor 26 may be tuned to recognize at least these two classes of hydraulic fluids and may be tuned to recognize others classes as well. There are four types of deicing fluids typically utilized in aviation applications, and the type utilized is based on the environmental conditions at the time of application. Deicing fluids may inadvertently be sprayed into the engines and APU inlets, which may cause the release of contaminants that have condensed onto heat exchangers, which would be detected by the gas sensor 26.

Referring now to the environmental parameters sensor 28, the sensor 28 may be provided to detect parameters such as motion, position, and barometric pressure, which are used to determine the flight profile of the aircraft to aid in delineating source contamination, and can account for a drop in sensor response plate voltage as altitude increases. In an implementation, the environmental parameters sensor 28 may include a three-axis geometric sensor and an inertial measurement unit. In another implementation, the environmental parameters sensor 28 may include a six-axis accelerometer and a magnetometer to determine changes in angle and acceleration and direction respectively. The sensors, along with pressure sensors, sense changes in cabin altitude to then make the determination the aircraft is airborne. A lack of motion, along with volatile organic compound and particle sensor responses, indicate contamination while the aircraft is on the ground. The stabilization of pressure at cruise cabin altitude provides input that can be used to set the baseline of the bleed air sensors at altitude, when the outside air is generally pristine and dry.

This sensing system 10 directly senses the bleed air in various aircraft locations before entering the cabin, such as high-pressure bleed ducts, air conditioning ducts, low pressure ducts downstream of the air conditioning pack, etc., and samples the air supplied by each engine and APU, at the exit of the ozone converters, and at the exit of the air conditioning pack allowing for the quantification of contaminants and to determine the source of contamination. Data enunciation to airline maintenance enables more ready isolation of contamination sources and allows for engine and APU quality checks to be performed on the air supply prior to shipping.

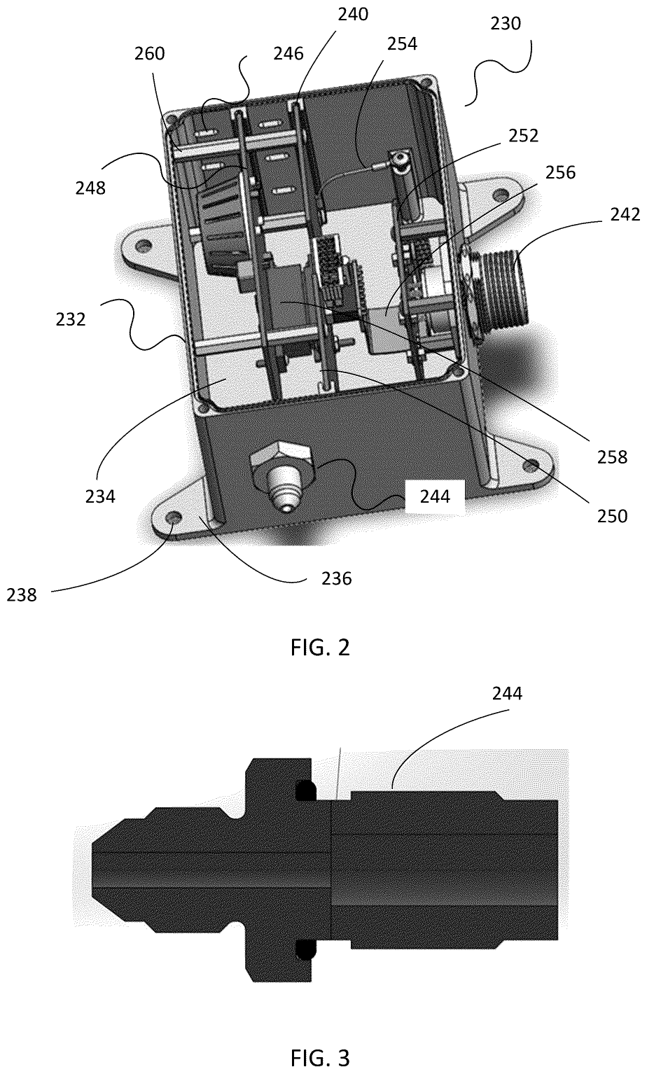

Turning now to FIG. 2, an exemplary sensor assembly or sensor packaging is shown at reference numeral 230. The assembly includes a housing 232 and cover (not shown) for closing the housing 232 and being sealed thereto. The housing 232 defines a cavity 234 and includes one or more feet 236, and as shown four feet extending from corners of the housing 232 with respective openings 238 for receiving fasteners for securing the housing 232 in the aircraft. The housing 232 also includes one or more channels 240 in the cavity 234 for receiving printed circuit boards (PCBs), an opening for a connector 242, an opening for one or more inlet pressure ports 244 to be coupled to the housing, and one or more vent holes 246 opposite the inlet pressure port 244 so air flows freely across a first PCB 248 that houses the sensing circuitry discussed above. The connector 242 is configured to connect to the aircraft, for example for connecting to a harness to provide power, communication lines, and analog lines to the assembly 230.

The inlet pressure port 244 and one or more vent holes 246 serve as the flow restrictor and vent respectively to create a pressure drop creating airflow through the housing 232 for a bleed air source that may be taken from various bleed air sources in the aircraft. The bleed air pressurizes the housing 232 and exhausts out of the one or more vent holes 246 at the opposite end of the housing so air flows freely across the first PCB 248. In an implementation, the housing 232 may be sealed around a second PCB 250 so that the air flows from the inlet pressure port 244 to the vent holes 246 and not to other portions in the housing 232. The one or more vent holes 246 may contain porous filters that block the ingestion of outside contaminants, particles and/or liquids which can contaminate the interior of the assembly 230. The filters may be hydrophobic polymer-based or sintered metal porous filter with hydrophobic coating, and the vent holes 246 may be sized to provide a known and stable airflow based on the input air pressure and flow profile of the selected filter. The internal temperature of the housing 232 may be monitored to adjust sensor response based on the internal sensor housing temperature.

The assembly further includes the second PCB 250 and a third PCB 252 positioned between the second PCB 250 and the connector 242. The second PCB 250 regulates power, has TVS diodes for lightning protection, and is grounded via wire 254 and the third PCB 252 has filter capacitors for electromagnetic interference (EMI) shunting. The second PCB 250 is connected to the third PCB 252 by a connector 256, such as a flexible ribbon cable, and a connector 258 is provided between the first PCB 248 and the second PCB 250 to provide clean power to the first PCB 248. One or more supports 260 may be provided to secure the PCB's 248, 250, and 252 to the housing 232 and/or one another.

The first PCB 248 is modular to allow for use in different aircraft locations containing different air sources at different velocities, while utilizing the same sensors for contaminant qualification. The different installation environment source pressures are to be scaled according to a cross-section of the inlet pressure port 244, shown in FIG. 3. The pressure values presented to the inlet pressure port 244, along with the physical geometry and mechanical features associated with the inlet pressure port, will determine the resulting airflow for each installation application. The left and right engine bleed location, for example, typically is nineteen to forty-five PSIG bleed air pressure, whereas APU bleed pressure is typically thirty-five to forty PSIG. At the exit of the left and right air condition pack, the pressure is typically fifteen to twenty in H2O. For proper ionization sensor operation, for example, the air entering the housing should travel at a sufficiently low velocity that the ionic fields in the sensor are not disrupted. Increasing air velocity approaching the ionic sensor causes a decrease in the voltage response on the sensing electrode with respect to the sealed reference electrode.

The cabin air sensing contains sufficient ventilation and relies on diffusion into and out of the housing 232. Alternatively, the unit is installed with a fan on the interior to pull air into the housing 232, which may be sized to provide air flow through the ionization sensor without disrupting the ionization field.

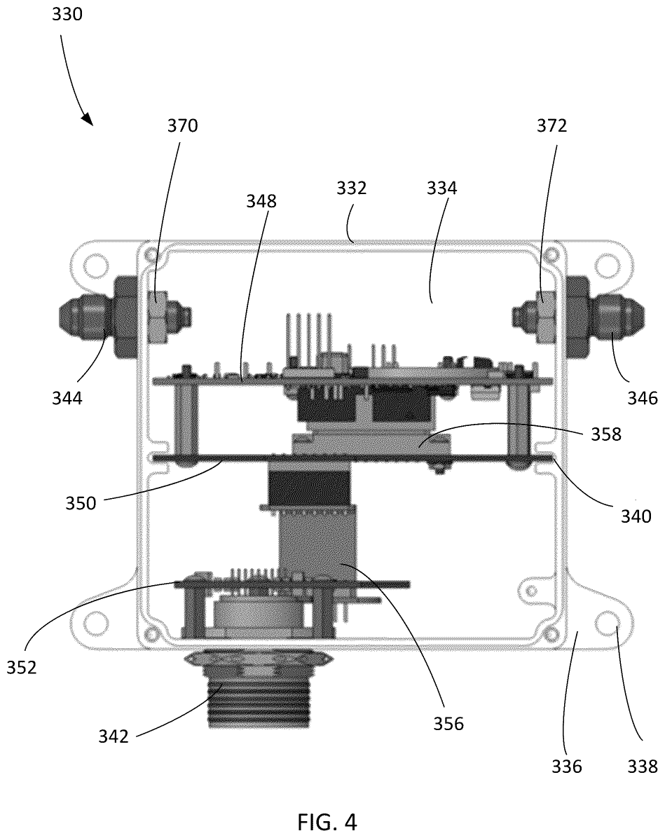

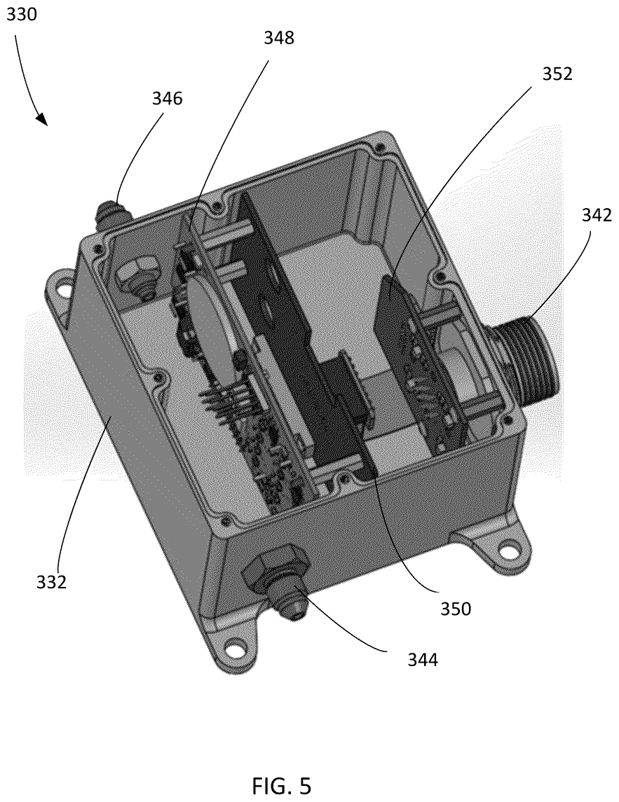

Turning now to FIGS. 4-6 an exemplary embodiment of the sensor assembly is shown at 330. The sensor assembly 330 is substantially the same as the above-referenced sensor assembly 230, and consequently the same reference numerals but indexed by 100 are used to denote structures corresponding to similar structures in the sensor assemblies. In addition, the foregoing description of the sensor assembly 232 is equally applicable to the sensor assembly 330 except as noted below. Moreover, the various sensors described below are substantially the same as those described in FIG. 1, and consequently the same reference numerals but indexed by 300 are used to denote structures corresponding to similar structures on the first PCB 348. It will also be appreciated that the sensors described herein may be used on the first PCB 248.

The sensor assembly 330 includes a housing 332 and cover (not shown) for closing the housing 332. The housing 332 defines a cavity 334 and includes one or more feet 336, and as shown four feet extending from corners of the housing 332 with respective openings 338 for receiving fasteners for securing the housing 332 in the aircraft. The housing 332 also includes one or more channels 340 in the cavity 234 for receiving PCBs, an opening for a connector 342, an opening for one or more inlet pressure ports 344, and an opening for one or more outlet pressure ports 346 opposite the inlet pressure port 344. The inlet and outlet pressure ports 344 and 346 may be coupled to the housing in a suitable manner, such as by respective nuts 370 and 372 threadably coupled to the inlet and outlet pressure ports 344 and 346 in the cavity 334. The inlet and outlet pressure ports 344 and 346 are sized to create a pressure drop creating airflow through the housing 332.

The assembly further includes a second PCB 350 and a third PCB 352 positioned between the second PCB 350 and the connector 342. The second PCB 350 is connected to the third PCB 352 by a connector 356, such as a flexible ribbon cable, and a connector 358 is provided between the first PCB 348 and the second PCB 350 to provide clean power to the first PCB 348.

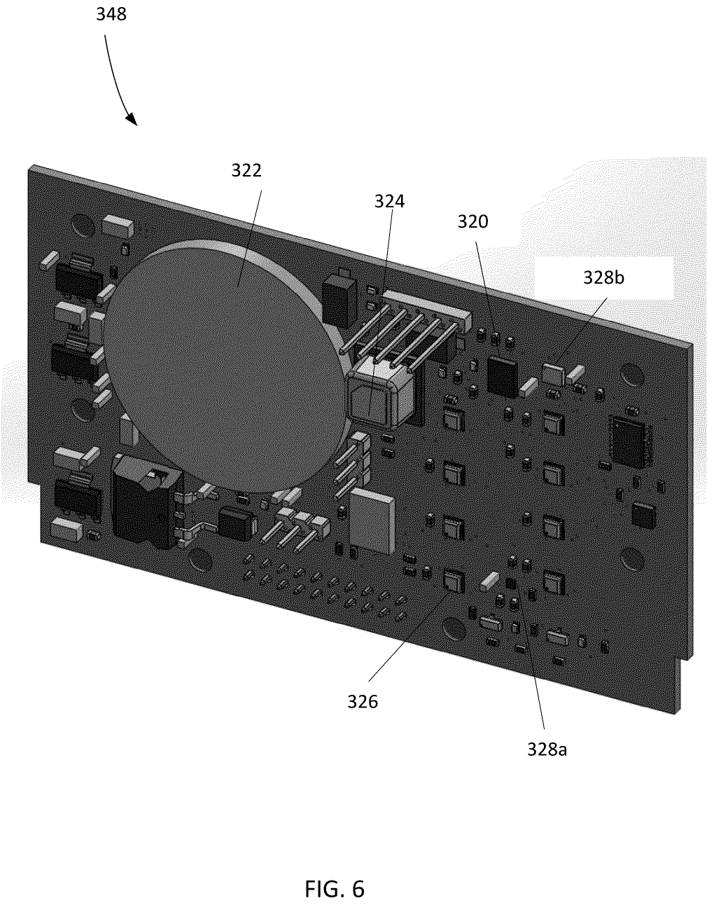

Turning now to FIG. 6, an implementation of the first PCB 348 will be described. The first PCB 348 houses the sensing circuitry discussed in FIG. 1 on a backside of the first PCB 348 for air to flow over the sensing circuitry. For example, the first PCB 348 houses a particulate matter sensor 320 such as an optical sensor, a particle sensor 322 such as an ion detector, a carbon dioxide sensor 324, at plurality of gas sensors 326 with artificial intelligence, and at least two environmental parameters sensors, such as a three-axis geometric sensor 328a and an inertial measurement unit 328b.

Although certain embodiments have been shown and described, it is understood that equivalents and modifications falling within the scope of the appended claims will occur to others who are skilled in the art upon the reading and understanding of this specification.

What is claimed is:

1. A sensing assembly comprising:
a housing defining a cavity;
a first printed circuit board (PCB) disposed in the cavity;
sensing circuitry disposed on the first PCB for detecting particles of various sizes;
an inlet in communication with the cavity upstream of the first PCB; and
an outlet in communication with the cavity downstream of the first PCB,
wherein the inlet and outlet are sized to create airflow across the first PCB.

2. The sensing assembly according to claim 1, wherein the sensing circuitry includes a particulate matter sensor, a particle sensor, a carbon dioxide sensor, a gas sensor, and an environmental parameters sensor.

3. The sensing assembly according to claim 2, wherein the particulate matter sensor is configured to detect and quantify particles down to about fifty nanometers, and the particle sensor is configured to detect and quantify particles down to about two nanometers.

4. The sensing assembly according to claim 2, wherein the environmental parameters sensor includes a three-axis geometric sensor and an inertial measurement unit.

5. The sensing assembly according to claim 2, wherein the gas sensor includes a plurality of gas sensors having artificial intelligence for identifying contaminant type.

6. The sensing assembly according to claim 2, wherein the sensing circuitry additionally includes a charge neutralizer.

7. The sensing assembly according to claim 1, further including a second PCB and a third PCB disposed in the housing, the second PCB being connected to the first PCB by a first connector and to the third PCB by a second connector.

8. The sensing assembly according to claim 7, wherein the second PCB regulates power to the first PCB and the third PCB has filter capacitors for electromagnetic interference shunting.

9. The sensing assembly according to claim 7, further comprising a harness connector coupled to the housing, the harness connector configured to be coupled to a harness to provide power, communication lines, and analog lines to the third PCB.

10. The sensing assembly according to claim 1, wherein the inlet is an inlet pressure port coupled to the housing, and the outlet is one or more vent holes in the housing.

11. The sensing assembly according to claim 1, wherein the inlet is an inlet pressure port coupled to the housing and the outlet is an outlet pressure port coupled to the housing.

12. A sensing assembly for use in an aircraft, the sensing assembly comprising:
a housing defining a cavity;
sensing circuitry disposed in the cavity for detecting particles of various sizes, the sensing circuitry includes a particulate matter sensor, a particle sensor, a carbon dioxide sensor, a gas sensor, and an environmental parameters sensor;
an inlet in communication with the cavity upstream of the sensing circuitry; and
an outlet in communication with the cavity downstream of the sensing circuitry,
wherein the inlet and outlet are sized to create airflow across the sensing circuitry.

13. The sensing assembly according to claim 12, further including a first printed circuit board (PCB) disposed in the cavity upon which the sensing circuitry is disposed, a second PCB disposed in the cavity and being connected to the first PCB by a first connector, and a third PCB disposed in the cavity and being connected to the second PCB by a second connector.

14. The sensing assembly according to claim 13, wherein the second PCB regulates power to the first PCB and the third PCB has filter capacitors for electromagnetic interference shunting.

15. The sensing assembly according to claim 13, further comprising a harness connector coupled to the housing, the harness connector configured to be coupled to a harness to provide power, communication lines, and analog lines to the third PCB.

16. The sensing assembly according to claim 12, wherein the environmental parameters sensor includes a three-axis geometric sensor and an inertial measurement unit, and wherein the gas sensor includes a plurality of gas sensors having artificial intelligence for identifying contaminant type.

17. The sensing assembly according to claim 12, wherein the inlet is an inlet pressure port coupled to the housing, and the outlet is one or more vent holes in the housing.

18. The sensing assembly according to claim 12, wherein the inlet is an inlet pressure port coupled to the housing and the outlet is an outlet pressure port coupled to the housing.

19. The sensing assembly according to claim 12, wherein the particulate matter sensor is configured to detect and quantify particles down to about fifty nanometers, and the particle sensor is configured to detect and quantify particles down to about two nanometers.

20. A method of sensing a source of contaminants from a bleed air source in an aircraft, such as pneumatic sources, temperature probe ports, gasper systems, mix manifolds, and return air filters, the method comprising:
receiving the bleed air at an inlet;
delivering the bleed air across one or more sensors, including a particulate matter sensor, a particle sensor, a carbon dioxide sensor, a gas sensor, and an environmental parameters sensor; and
delivering the bleed air to an outlet.
